(19) United States
(12) Patent Application Publication
(10) Pub. No.: US 2008/0038359 A1
(43) Pub. Date: Feb. 14, 2008

(54) Stable Nanoparticle Formulations

(75) Inventor: Khawla Abdullah ABU-IZZA, Highland Mills, NY (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS U.S. LLC, Bridgewater, NJ (US)

(21) Appl. No.: 11/874,393

(22) Filed: Oct. 18, 2007

Related U.S. Application Data
(63) Continuation of application No. PCT/US2006/017059, filed on May 3, 2006.
(60) Provisional application No. 60/678,086, filed on May 5, 2005.

(51) Int. Cl. A61K 9/14

(52) U.S. Cl. 424/489; 977/906

(57) ABSTRACT

The invention relates to pharmaceutically stable nanoparticle formulations of poorly soluble drug substances, to the processes for the preparation of such formulations, and to methods of use thereof.

The present invention relates to pharmaceutically stable nanoparticle formulations of poorly soluble drug substances. This invention also relates to processes for the preparation of such formulations.

### BACKGROUND OF THE INVENTION

A major problem in formulating many biologically active compounds is their poor solubility or insolubility in water. Oral formulations of water-insoluble or poorly soluble biologically active agents frequently show poor and erratic bioavailability. Consequently, small particle formulations of drugs are often needed to maximize the surface area and, therefore, the bioavailability and dissolution rate of the active agent. Compositions containing nanoparticles of drug substances, that is particles generally having an average size of less than about 1000 nanometers (nm), in suspensions have shown success in increasing the bioavailability of poorly soluble drug substances.

It is desirable that a water-insoluble or poorly soluble active substance be stable when formulated. For any suspension, and for a nanoparticle suspension (nanosuspension) in particular, there are two destabilizing processes against which the suspension needs to be stabilized. These processes are particle aggregation or flocculation and particle growth by Ostwald ripening, resulting from changes in solubility due to temperature fluctuation during storage. The more temperature sensitive the drug solubility is, the more susceptible the suspension will be to particle growth by Ostwald ripening. Conventional nanosuspensions (generally, suspensions in liquid vehicles) are stabilized against aggregation by surface modifiers (for example, nonionic surfactants or polymers) that are adsorbed onto the drug particle surface. Such surface modifiers stabilize the drug particles as a result of repulsion due to the steric interaction between the polymeric chains of surface modifiers protruding from the drug particles' surfaces. This effect depends on the nature, thickness and completeness of the surfactant/polymer adsorbed layers on the particles. The surface modifier may also stabilize against crystal growth, wherein the polymer or nonionic surfactant forms a net-like film on the crystal surface, allows the crystal to grow out only through the openings of the net, and, hence, slows down crystal growth. The more condensed and the less porous the film is, the better are its barrier properties and its ability to stabilize the particles against crystal growth. Nevertheless, certain nanoparticle suspension formulations can be susceptible to active agent particle aggregation even when a surface stabilizer or modifier is present, such as when the formulation is heated to temperatures above the cloud point of the surface stabilizer. At temperatures above their cloud point, surface stabilizers dissociate from the nanoparticles and precipitate, leaving the nanoparticles unprotected. The unprotected nanoparticles may then aggregate into clusters of particles. Upon cooling, the surface stabilizers may redissolve into the solution and then coat the aggregated particles and prevent them from dissociating into more desirable smaller particles.

Accordingly, it is the object of the present invention to provide stable nanoparticle formulations that do not require the use of a surface modifier or stabilizer.

### SUMMARY OF THE INVENTION

The present invention relates to pharmaceutically stable nanoparticle formulations comprising particles of a poorly soluble drug substance having an average particle size of less than about 1000 nm and a solid or semisolid dispersion vehicle.

The present invention also provides processes for preparing the stable nanoparticle formulations of the instant invention and to methods of use thereof.

### DETAILED DESCRIPTION OF THE INVENTION

A key feature of the nanoparticle formulations of the present invention is their ability to be stable without having a surface modifier or stabilizer adsorbed onto the surface of the drug particles. Unlike conventional nanosuspensions, the stabilization mechanism of the formulations of the present invention does not involve a surface phenomenon. The solid or semisolid vehicle acts as a stabilizer against aggregation by a physical effect on the mobility of the drug particles. When the vehicle forms a solid at room temperature, or has a consistency of a very viscous semisolid, it stops or slows down the drug particles' movement and, hence, prevents the drug particles from aggregating. It is generally known that the stability of a suspension increases with the viscosity of the vehicle or dispersion medium. The solid or semisolid vehicle also forms a physical barrier against particle growth. The closely packed structure of a non-porous solid or semisolid matrix does not provide the crystals with space to grow. Additionally, the solubility of the drug substance in a solid or semisolid matrix is less sensitive to small changes in temperature than the solubility of a drug substance in a liquid vehicle, and therefore, the semisolid or solid suspension is less susceptible to crystal growth by Ostwald ripening.

A “poorly soluble drug substance” of the present invention is a drug substance having poor solubility in water, that is, less than about 10 mg/ml at physiological pH (2-7.5). Preferably the water solubility of the drug substance is less than about 5 mg/ml, more preferably less than about 1 mg/ml, and most preferably less than about 0.1 mg/ml. The drug substance is suspended within the dispersion vehicle or matrix at molten temperatures. Therefore, a poorly soluble drug substance, as used herein, also has poor solubility in the dispersion vehicle at molten temperatures (that is, temperatures at or above the melting point of the solid or semisolid dispersion vehicle). Preferably, the drug substance solubility in the molten dispersion matrix is less than about 3 mg/g, more preferably less than about 1 mg/g, and most preferably less than about 0.5 mg/g.

In a preferred embodiment, the nanoparticle formulations of the present invention contain poorly soluble drug substance nanoparticles having an average particle size of less than about 1000 nm, preferably less than about 750 nm, more preferably less than about 600 nm, and, in particular, less than about 500 nm. In another preferred embodiment, the nanoparticle formulations of the present invention contain poorly soluble drug substance in which at least 90%, and more preferably at least 95%, of the drug particles have a particle size less than about 1000 nm.

The amount of poorly soluble drug substance in the nanoparticle formulations of the present invention ranges from about 0.001% to about 30% by weight. In a preferred embodiment the amount of poorly soluble drug substance ranges from about 1% to about 20% by weight.

The nanoparticle formulations of the present invention preferably contain a therapeutically effective amount of the poorly soluble drug substance(s). The term “therapeutically effective amount,” as used herein, refers to an amount of the drug substance present in the formulation being administered that is sufficient to prevent development of or alleviate to some extent one or more of the symptoms of the disease being treated. Likewise, a therapeutically effective amount of a nanoparticle pharmaceutical formulation refers to an amount of such formulation that is sufficient to prevent development of or alleviate to some extent one or more of the symptoms of the disease being treated. In determining the effective amount or dose, a number of factors are considered by the attending diagnostician, including, but not limited to: the species of mammal; its size, age, and general health; the specific disease involved; the degree of involvement or the severity of the disease; the response of the individual patient; the particular compound administered; the mode of administration; the bioavailability characteristics of the preparation administered; the dose regimen selected; the use of concomitant medication; and other relevant circumstances.

Suitable drug substances can be selected from a variety of known classes of drugs including, for example, proteins, peptides, nutraceuticals, anti-inflammatory agents, NSAIDS, COX-2 inhibitors, analgesics, antimuscarinic and muscarinic agents, corticosteroids, elastase inhibitors, oncology therapies, antiemetics, neuroprotection agents, cardiovascular agents, anti-platelet agents, lipid regulating agents, anticoagulants, anthelmintics, antiarrhythmic agents, cardiac inotropic agents, antihypertensive agents, diuretics, diagnostic agents, diagnostic imaging agents, antiviral agents, anti-fungals, antibiotics, antimycobacterial agents, anticonvulsant agents, antidiabetic agents, antiepileptics, antineoplastic agents, immunoactive agents, immunosuppressive agents, antithyroid agents, thyroid agents, antidepressants, anesthetics, anxiolytic agents, hypnotics, neuroleptics, astringents, beta-andrenoceptor blocking agents, dopaminergics, haemostatics, immuriological agents, muscle relaxants, parasympathomimetics, parathyroid calcitonin, biphosphonates, prostaglandins, radio-pharmaceuticals, steroids, sex hormones, stimulants and anoretics, sympathomimetics, anti-allergic agents, antihistamines, cough suppressants, vasodilators, and xanthines. A detailed description of these and other suitable drugs may be found, for example, in Martindale, The Extra Pharmacopoeia, 31^{st} Edition (The Pharmaceutical Press, London, 1996), the disclosure of which is hereby incorporated by reference in its entirety. The drug substances are commercially available and/or can be prepared by techniques known in the art.

Examples of preferred poorly soluble drugs for the purposes of the present invention include 7-chloro-N,N,5-trimethyl-4-oxo-3-phenyl-3,5-dihydro-4H-pyridazino[4,5-b]indole-1-acetamide, 6-fluoro-9-methyl-2-phenyl-4-(pyrrolidin-1-ylcarbonyl)-2,9-dihydro-1H-pyrido[3,4-b]indol-1-one, and isopropyl 2-butyl-3-[4-[3-(dibutylamino)propyl]benzoyl]-1-benzofuran-5-carboxylate fumarate.

7-Chloro-N,N,5-trimethyl-4-oxo-3-phenyl-3,5-dihydro-4H-pyridazino[4,5-b]indole-1-acetamide (hereinafter “Compound A”) has the following chemical structure: 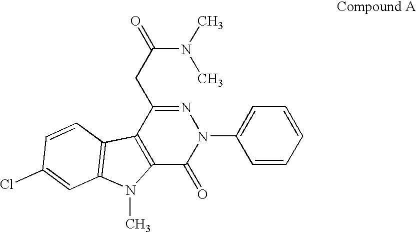
Compound A is useful as, for example, a neuroprotective agent for the treatment of neurodegenerative diseases or as an oncology therapeutic for the treatment of cancer, and can prepared according to the basic procedures described in U.S. Pat. No. 6,262,045 and, in particular, U.S. Pat. No. 6,395,729, which patents are incorporated by reference herein.

6-Fluoro-9-methyl-2-phenyl-4-(pyrrolidin-1-ylcarbonyl)-2,9-dihydro-1H-pyrido[3,4-b]indol-1-one (hereinafter “Compound B”) has the following chemical structure: 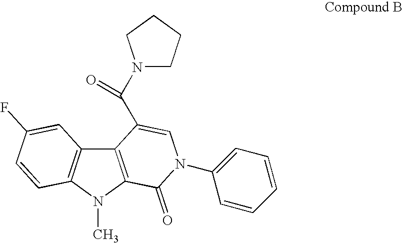
Compound B is useful as, for example, an anxiolytic agent for the treatment of anxiety or as an anticonvulsant agent for the treatment of epilepsy, spasticity, or muscle contractures; and can prepared according to the basic procedures described in U.S. Pat. No. 6,075,021, which patent is incorporated by reference herein.

Isopropyl 2-butyl-3-[4-[3-(dibutylamino)propyl]benzoyl]-1-benzofuran-5-carboxylate fumarate (hereinafter “Compound C”) has the following chemical structure: 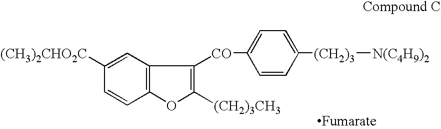
Compound C is useful, for example, for the treatment or prevention of arrhythmia as an antiarrhythmic, and can prepared according to the basic procedures described in U.S. Patent Application Publication No. 2003/0225100, published Dec. 4, 2003, and WO 02/16339, published Feb. 28, 2002, which references are incorporated by reference herein.

In the nanoparticle formulations of the present invention, the drug substance can be either in a crystalline state or amorphous depending on the drug substance and drug concentration.

The dispersion vehicle of the present invention is a non-surface modifying material (that is, a material which does not adsorb onto the surface of the drug particle), suitable for pharmaceutical preparations, such as for oral and/or topical applications, which is a solid or semisolid at ambient temperatures, but melts above room temperature. Preferred dispersion vehicles are those that melt between about 30° C. and about 110° C. Other preferred vehicles are those that melt between about 30° C. and about 80° C., and more preferably between about 35° C. and about 60° C. The dispersion vehicle can be a single material having the previously described properties, or a mixture of materials (such as, for example, an oil and a wax) that when combined become a solid or semisolid at ambient temperature and melt preferably below about 110° C., more preferably below about 80° C., and most preferably below about 60° C.

A semisolid dispersion vehicle, as used herein, is a material, or mixture of materials that when combined have rheological properties of both a liquid and a solid. When standing, they have a high consistency and do not begin to flow until a force is applied (for example, by mixing, spreading, or extruding), which exceeds a minimum value of force, known as the “yield value,” which is characteristic of a given semisolid. After the yield value is exceeded, semisolids behave more like liquids; semisolids flow, whereas solids deform when shear stress exceeding their yield value is applied. The viscosity of semisolids is dependant on the shear rate. Preferred semisolid vehicles are shear thinning, that is, their viscosity decreases as the shear increases. The viscosity also decreases with temperature; a material could be a solid at room temperature (deforms under shear stress above the yield value) and semisolid at elevated temperature (flows under shear stress above the yield), or a semisolid at room temperature and a liquid at elevated temperature.

A semisolid dispersion vehicle of the present invention can optionally contain a number of materials to produce the desired consistency and texture profile. In an ointment-like semisolid, all of the materials in the vehicle are miscible (single phase vehicle), as, for example, a vehicle composed of mineral oil and petrolatum or a miscible wax such as paraffin wax, which can be used, for example, for topical dosage forms. Examples of preferred oral semisolid dispersion vehicles include mixtures of a vegetable oil (for example, soybean oil) or medium chain triglycerides with one or more of the following: high melting point hydrogenated vegetable oil (vegetable stearine), ester(s) of long-chain fatty acid(s) such as glyceryl behenate (commercially known as Compritol®), and/or an edible wax, for example, castor wax or beeswax.

Additional examples of preferred dispersion vehicles include hydrogenated vegetable oils (such as Wecobee® S available from Stepan Company, Northfield, Ill., and Hydrokote™ 112 available from Abitec Corporation, Columbus, Ohio); triglycerides, for example hydrogenated coco-glycerides (such as Softisan® 142, available from Sasol Inc.); mixed glycerides; hydrogenated glycerides; synthetic glycerides; glycerin esters of fractionated fatty acids; non-surface active esters of fatty acids, for example propylene glycol diesters of fatty acids; fatty acids, such as stearic acid and palmitic acids; cocoa butter and cocoa butter substitutes; hard fat (such Softisan® 154, available from Sasol Inc.); natural and synthetic waxes; and petrolatum.

Nanoparticle formulations according to the present invention may optionally include additional non-surface modifying excipients generally used in the art. Such excipients may include one or more fillers, sweeteners, flavoring agents, colorants, preservatives, buffers, and other excipients depending on the route of administration and the dosage form used.

The formulations of the present invention are generally administered to patients, which include, but are not limited to, mammals, for example, humans, by conventional routes known in the art. For example, the formulations can be administered to patients orally, in the form of, for example, a hard or soft gelatin capsule, a tablet, a caplet, or a suspension; rectally or vaginally, for example in the form of a tablet, suppository or pessary, paste, ointment, lotion, or suspension; or topically, for example in the form of a paste, ointment, lotion or suspension.

Preferred embodiments of the present invention include nanoparticle formulations comprising a poorly soluble drug substance and a semisolid dispersion vehicle for topical administration in the form of an ointment or paste.

The present invention further relates to the use of the nanoparticle formulations of the invention in medicine.

In another embodiment, the invention relates to a method of treating a patient, for example, a mammalian patient such as a human patient, with a nanoparticle pharmaceutical formulation of the present invention, the method involving administering an effective amount of a nanoparticle formulation of the invention to the patient.

A preferred method of the invention relates to a method of treating a neurodegenerative disease or cancer, which comprises administering to a patient in need of such treatment a therapeutically effective amount of a nanoparticle pharmaceutical formulation of the present invention in which the poorly soluble drug substance is 7-chloro-N,N,5-trimethyl-4-oxo-3-phenyl-3,5-dihydro-4H-pyridazino[4,5-b]indole-1-acetamide.

Another preferred method of the invention is a method of treating or preventing anxiety, epilepsy, spasticity, or muscle contractures, which comprises administering to a patient in need of such treatment or prevention a therapeutically effective amount of a nanoparticle pharmaceutical formulation of the present invention in which the poorly soluble drug substance is 6-fluoro-9-methyl-2-phenyl-4-(pyrrolidin-1-ylcarbonyl)-2,9-dihydro-1H-pyrido[3,4-b]indol-1-one.

Another preferred method of the invention is a method of treating or preventing arrhythmia, which comprises administering to a patient in need of such treatment or prevention a therapeutically effective amount of a nanoparticle pharmaceutical formulation of the present invention in which the poorly soluble drug substance is isopropyl 2-butyl-3-[4-[3-(dibutylamino)propyl]benzoyl]-1-benzofuran-5-carboxylate fumarate.

A subject of the present invention is the use of the nanoparticle formulation of the present invention wherein the poorly soluble drug substance is 7-chloro-N,N,5-trimethyl-4-oxo-3-phenyl-3,5-dihydro-4H-pyridazino[4,5-b]indole-1-acetamide in the manufacture of medicinal products for the treatment of a neurodegenerative disease or cancer.

A further subject of the invention includes the use of the nanoparticle formulation of the present invention wherein the poorly soluble drug substance is 6-fluoro-9-methyl-2-phenyl-4-(pyrrolidin-1-ylcarbonyl)-2,9-dihydro-1H-pyrido[3,4-b]indol-1-one in the manufacture of medicinal products for the treatment of anxiety, epilepsy, spasticity, or muscle contractures.

A further subject of the invention includes the use of the nanoparticle formulation of the present invention wherein the poorly soluble drug substance is isopropyl 2-butyl-3-[4-[3-(dibutylamino)propyl]benzoyl]-1-benzofuran-5-carboxylate fumarate in the manufacture of medicinal products for the treatment of arrhythmia.

In another embodiment, the present invention relates to dosage forms comprising the nanoparticle formulation described herein. Dosage forms include, but are not limited to, those selected from the group consisting of pills, capsules, caplets, tablets, granules, suspensions, ointments, lotions, suppositories, and pastes.

It will also be apparent to those skilled in the art that the formulations of the present invention can be administered with other therapeutic and/or prophylactic agents and/or medicaments that are not medically incompatible therewith.

All components of the present formulations must be pharmaceutically acceptable. As used herein, a “pharmaceutically acceptable” component is one that is suitable for use with humans and/or other animals without undue adverse side effects (such as toxicity, irritation and allergic response) commensurate with a reasonable benefit/risk ratio.

The present invention further relates to a process for preparing nanoparticle formulations of the present invention which comprises mixing a poorly soluble drug substance with a molten dispersion vehicle which is a solid or semi-solid at room temperature, and media-milling the mixture to form a nanoparticle formulation.

A preferred process for preparing the nanoparticle formulations of the present invention comprises the steps of heating a solid or semisolid dispersion vehicle to a temperature range at or above the melting point of the dispersion vehicle to form a molten dispersion vehicle; combining one or more poorly soluble drug substance(s) with the molten dispersion vehicle to form a mixture; media milling the mixture with a plurality of grinding media to form a nanosuspension; and cooling the nanosuspension to a temperature range below the melting point of the dispersion vehicle.

In a particularly preferred process for preparing the nanoparticle suspension formulations of the present invention, the media milling step is performed at temperatures only slightly higher than the melting point of the dispersion vehicle. Preferably, the process is carried out at less than about 10° C. above the melting point of the dispersion vehicle, and more preferable less than about 5° C. above the melting point of the dispersion vehicle.

Another aspect of the instant invention involves the step of filling the milled nanosuspension into capsules prior to cooling the suspension to a temperature below the melting point of the dispersion vehicle. In an additional aspect of the present invention, the milled nanosuspensions, prior to cooling, are granulated directly onto one or more non-surface modifying pharmaceutically acceptable solid filler(s) generally used in the art, such as, for example, lactose, mannitol, and cornstarch, to generate a solid granulation formulation with no drying step.

Media milling is a process well known to those skilled in the art for preparing nanoparticle suspensions. The process is preferably carried out in a mill, such as a cylindrical vessel, having a milling chamber containing a plurality of grinding media, the drug substance which is to be milled, and the dispersion vehicle in which the grinding media and drug substance are suspended. The milling chamber may optionally contain additional non-surface modifying excipients. The chamber is maintained at or slightly above the melting temperature of the dispersion vehicle. The contents of the milling chamber are stirred or agitated with an agitator which transfers energy to the grinding media. The accelerated grinding media collide with the drug substance in energetic collisions that can crush, chip, fracture or otherwise reduce the size of the solid substrate material and lead to an overall reduction in drug particle size and an overall reduction in drug average or mean particle size distribution. A sieve or screen at the outlet holds the grinding medium back.

In a preferred process of the invention, the grinding media, the dispersion vehicle, and the drug substance being milled remain in the vessel until the fractured drug substance particles have been reduced to the desired size or to a minimum size achievable. The nanosuspensions (that is, the drug particles suspended in the dispersion vehicle) are then separated from the grinding media with a separator or screen at the outlet of the milling chamber.

In another preferred process of the invention, the milling process occurs in a recirculating manner (continuous mode). A mill operating in a recirculating manner incorporates a separator or screen for retaining grinding media together with relatively large particles of the drug substance being milled in the milling chamber, while allowing smaller particles of the drug substance being milled to pass out of the milling chamber. Recirculation often involves a suspension which moves from the milling chamber into a holding vessel and then back to the milling chamber, frequently with the aid of a pump. A separator or screen can be located at the outlet of the milling chamber.

In a third preferred process of the invention, the milling process occurs in discrete passes (discontinuous mode). In discontinuous mode, a mixture of the drug substance and the dispersion vehicle is pumped through the milling chamber and then into a separate receiving container, constituting a single pass. This process may be repeated until the desired particle size is achieved.

Grinding media are generally spherical or cylindrical beads selected from a variety of dense and hard materials, such as, for example, sand, steel, silicon carbide, ceramics, zirconium silicate, zirconium and yttrium oxide, glass, alumina, titanium, and certain polymers such as crosslinked polystyrene and methyl methacrylate. Possible metal contamination from metal grinding medium, such as zirconium, may be reduced by preconditioning the grinding media in blank dispersion vehicle to allow any initial attrition to occur prior to the addition of the drug suspension into the mill.

As used herein with reference to stable nanoparticle formulations, “stable” refers to a nanoparticle formulation in which the drug particles do not appreciably flocculate or agglomerate due to interparticle attractive forces, or otherwise significantly increase in particle size over time; the drug particles have chemical stability; and/or the physical structure of the drug particles does not alter over time, such as by conversion from an amorphous phase to a crystalline phase.

The following examples will further illustrate the invention, without, however, limiting it thereto.

### EXAMPLE 1

### Compound A in Hydrogenated Vegetable Oil

The solubility of Compound A in the proposed dispersion vehicle, Wecobee® S (a triglyceride derived from vegetable oils, melting point 44° C.), was initially determined by the following process. 5 grams of the hydrogenated vegetable oil were weighed into a scintillation vial and heated to 50° C. 5 g of Compound A was added and stirred in a water bath on a magnetic stirrer. The drug substance did not dissolve, so additional hydrogenated vegetable oil was added gradually until the total amount of the hydrogenated vegetable oil was 10 g. The mixture was left stirring overnight in a water bath at 60° C. The mixture was filtered at the same temperature (the filtration assembly was heated in an oven to 60° C.), and the solubility of the filtrate for Compound A in the hydrogenated vegetable oil at 60° C. was determined to be 0.48 mg/g.

To prepare the suspension formulation, the following process was utilized: 250 ml of 1.0 mm Yttrium-stabilized zirconia beads were loaded into a DynoMill (type KDL 0.3 L SS milling chamber, available from Glen Mills). Initially, the circulating water bath temperature (which controls the temperature of the seal area) and the tap water temperature (which controls the temperature of the milling chamber) were set at 50° C. to heat the chamber and the beads. For initial washing and conditioning of the beads, soybean oil was circulated at 40 ml/min with the agitator stirring at 3200 rpm. After a few minutes, the circulating water bath temperature was lowered to 40° C. to keep the temperature cooled below 60° C. The tap water temperature was also adjusted to 45° C. Following soybean oil, molten hydrogenated vegetable oil (Wecobee® S) was circulated for further conditioning and to washout the liquid oil. The total conditioning and washing time (soy oil and Wecobee® S) was approximately one hour.

The drug substance suspension was prepared by dispersing 150 g of Compound A in 700 g of molten Wecobee® S at 50° C. using an overhead mixer (Lightnin® brand) on a hotplate. After draining the washing vehicle from the milling equipment, the drug substance suspension was circulated at 400 ml/min with the DynoMill™ stirring at 3200 rpm. The suspension was kept stirring using the mixer to prevent sedimentation, but was not heated during milling. The circulating water bath temperature and the circulating tap water temperature were lowered further to cool and maintain the temperature around 55° C. and the product temperature between 45° C. and 50° C. These temperatures were maintained throughout the milling period. The suspension was milled for a total of 5 hours. After the end of the milling, the suspension was transferred to a storage container and allowed to cool to room temperature.

### EXAMPLE 2

### Compound B in Hard Fat

The solubility of Compound B in soy oil was initially estimated visually by gradual addition of soy oil to a weighed amount of the drug substance until the oil was almost clear (visually). An estimate of the drug substance in oil was calculated from the total amount of oil added. The solubility of Compound B in soy oil was less than 1 mg/ml, and, thus, it was reasoned that the low solubility of Compound B in soy oil would translate into a low solubility in hard fat as well.

This suspension formulation was prepared using a vertical mill with hard fat (Softisan® 154, a hydrogenated palm oil with melting point range of about 53-58° C.). The hard fat was melted by heating on a hot plate. 1.0 mm Yttrium-stabilized zirconia beads (20 ml) were preheated in a 50 ml plastic tube to 50° C. 2 g of Compound B, followed by 10 ml of the molten hard fat were added. A heating tape was wrapped around the upper part of the tube to keep the contents molten. The formulation was stirred at 2000 rpm for 3 hours using the vertical mill. Due to the relatively high melting point of the hard fat, it was necessary to continue heating with the heating tape until the end of the milling period. Attempts to stop the heating resulted in solidification of the fat on the upper part of the tube. At the end of the milling process, the molten suspension was screened to remove the milling beads.

### EXAMPLE 3

### Compound C in Hydrogenated Coco-Glycerides

The vehicle (hydrogenated coco-glycerides, commercially known as Softisan® 142, melting point range of about 42-44° C.) was heated to 50° C. on a hotplate. 2.5 g of Compound C, which was generally known to have a low solubility in oils, were weighed into a 50-ml centrifuge tube, and the tube was heated in a convection oven to 50° C. 20 ml of 1 mm very high-density zirconium beads were heated in another tube to the same temperature. The beads were added to the drug substance, followed by the addition of 10 ml of Softisan® 142. A heating tape was wrapped around the upper part of the tube, and the temperature control was set on low. The vertical mill impeller was inserted in the tube, and the formulation was mixed at 2000 rpm for 3 hours. An additional 10 ml of molten Softisan® 142 were added and mixed with a glass rod. The molten suspension was then screened at 55° C. to remove the milling beads using a preheated filtration assembly.

### EXAMPLE 4

### Particle Size Analysis of Examples 1 to 3

Particle size analysis for Examples 1 to 3 was performed using a Horiba LA-920 Laser diffraction particle sizer. A 200 mg portion of sample was first heated in a water bath at 50° C., 25 ml of soybean oil containing Aerosol® OT-100 dispersant (sodium dioctyl-sulfosuccinate available from Cytec Industries Inc.) was added, and then the sample was stirred. The sample was transferred to the instrument, stirred, sonicated, and analyzed. The results for the particle size analysis of Examples 1 to 3 are provided in Tables 1A, 1B, and 1C, below.

TABLE 1A
Particle Size Distribution of Example
1, Compound A in Wecobee ® S

| Median Particle Size | 369 nm |
|---|---|
| Mean Particle Size | 458 nm |
| Percent below 1 micron | 96.0% |
| Percent below 500 nm | 77.2% |

TABLE 1B
Particle Size Distribution of Example 2,
Compound B in Softisan ® 154

| Median Particle Size | 236 nm |
|---|---|
| Mean Particle Size | 242 nm |
| Percent below 1 micron | 100% |
| Percent below 500 nm | 99.7% |

TABLE 1C
Particle size distribution of Example 3,
Compound C in Softisan ® 142

| Median | 482 nm |
|---|---|
| Mean | 508 nm |
| Percent below 1 micron | 98.6% |
| Percent below 500 nm | 57.0% |

The above-results demonstrate that the process of the present invention can be utilized to prepare nanoparticle formulations, in which the drug particles have an average particle size of less than 1000 nm.

### EXAMPLE 5

### Physical Stability of Example 1

To determine the physical stability of the nanoparticle suspension prepared according to Example 1, the suspension was stressed for three months at 40° C./75% relative humidity (RH). The sample was analyzed for particle size stability at the end of each of three months.

Table 2 lists the particle size parameters of the stressed samples in comparison with those at the initial time point.

TABLE 2
Particle size stability of Example 1

| Parameter | Time 0 | 1 Month | 2 Months | 3 Months |
|---|---|---|---|---|
| Median | 369 nm | 368 nm | 358 nm | 377 nm |
| Mean | 458 nm | 460 nm | 442 nm | 474 nm |
| Percent below | 96.0% | 96.0% | 96.9% | 96.0% |
| 1 micron | | | | |
| Percent below | 77.2% | 79.5% | 82.4% | 77.5% |
| 500 nm | | | | |

### EXAMPLE 6

### Physical Stability of Example 2

To determine the physical stability of the composition prepared according to Example 2, the suspension was stressed by alternating heating and cooling; a sample was stored at 50° C. for one week, at 5° C. for the second week and at 50° C. for the third week. The sample was analyzed for particle size stability at the end of three weeks.

Table 3 compares the particle size distribution of the suspension of Example 2 initially and at the end of the three weeks of heat/cool stressing.

TABLE 3
Particle Size Stability of Example 2 after three weeks of stressing

| | | Stressed for 3 |
|---|---|---|
| Parameter | Initial | weeks |
| Median | 236 nm | 242 nm |
| Mean | 242 nm | 267 nm |
| Percent below 1 micron | 100% | 100% |
| Percent below 500 nm | 99.7% | 95.4% |

### EXAMPLE 7

### Physical Stability of Example 3

To determine the physical stability of the nanoparticle formulation prepared according to Example 3, the formulation was stressed for two weeks at 50° C. Table 4 compares the particle size distribution of the formulation of Example 3 initially and at the end of the two weeks of stressing.

TABLE 4
Particle size stability of Example 3formulation
after two weeks at 50° C.

| | | Stressed for 2 |
|---|---|---|
| Parameter | Initial | weeks |
| Median | 482 nm | 492 nm |
| Mean | 508 nm | 514 nm |
| Percent below 1 micron | 98.6% | 98.8% |
| Percent below 500 nm | 54.7% | 52.3% |

What is claimed is:

1. A nanoparticle pharmaceutical formulation comprising a poorly soluble drug substance having an average particle size of less than about 1000 nm, a solid or semisolid dispersion vehicle, and optionally a non-surface modifying excipient.

2. The formulation according to claim 1 wherein said poorly soluble drug substance has an average particle size of less than about 750 nm.

3. The formulation according to claim 1 wherein said poorly soluble drug substance has an average particle size of less than about 600 nm.

4. The formulation according to claim 1 wherein at least 95% of the poorly soluble drug substance has a particle size less than about 1000 nm.

5. The formulation according to claim 1, wherein the amount of poorly soluble drug substance in the formulation ranges from about 0.01% to about 30% by weight.

6. The formulation according to claim 5, wherein the amount of poorly soluble drug substance in the formulation ranges from about 1% to about 20% by weight.

7. The formulation according to claim 1, wherein the non-surface modifying excipient is a non-surface modifying pharmaceutically acceptable solid filler.

8. The formulation according to claim 1 wherein said poorly soluble drug substance is one or more selected from the group consisting of a protein, a peptide, a nutraceutical, an anti-inflammatory agent, an NSAID, a COX-2 inhibitor, an analgesic, an antimuscarinic agent, a muscarinic agent, a corticosteroid, an elastase inhibitor, an oncology therapy agent, an antiemetic, a neuroprotection agent, a cardiovascular agent, an anti-platelet agent, a lipid regulating agent, an anticoagulant, an anthelmintic, an antiarrhythmic agent, a cardiac inotropic agent, an antihypertensive agent, a diuretic, a diagnostic agent, a diagnostic imaging agent, an antiviral agent, an anti-fungal agent, an antibiotic, an antimycobacterial agent, an anticonvulsant agent, an antidiabetic agent, an antiepileptic, an antineoplastic agent, an immunoactive agent, an immunosuppressive agent, an antithyroid agent, a thyroid agent, an antidepressant, an anesthetic, an anxiolytic agent, a hypnotic, a neuroleptic, an astringent, a beta-andrenoceptor blocking agent, a dopaminergic, a haemostatic, an immuriological agent, a muscle relaxant, a parasympathomimetic, a parathyroid calcitonin, a biphosphonate, a prostaglandin, a radio-pharmaceutical, a sex hormone, a steroid, a stimulant, an anoretic, a sympathomimetic, an anti-allergic agent, an antihistamine, a cough suppressant, a vasodilator, and a xanthine.

9. The formulation according to claim 8 wherein said poorly soluble drug substance is one or more selected from the group consisting of a neuroprotection agent, an antiarrhythmic agent, an anticonvulsant agent, and an anxiolytic agent.

10. The formulation according to claim 1 wherein said poorly soluble drug substance is selected from the group consisting of 7-chloro-N,N,5-trimethyl-4-oxo-3-phenyl-3,5-dihydro-4H-pyridazino[4,5-b]indole-1-acetamide, 6-fluoro-9-methyl-2-phenyl-4-(pyrrolidin-1-ylcarbonyl)-2,9-dihydro-1H-pyrido[3,4-b]indol-1-one, and isopropyl 2-butyl-3-[4-[3-(dibutylamino)propyl]benzoyl]-1-benzofuran-5-carboxylate fumarate.

11. The formulation according to claim 1 wherein said dispersion vehicle is one or more material(s) selected from the group consisting of hydrogenated vegetable oils, triglycerides, hydrogenated coco-glycerides, mixed glycerides, hydrogenated glycerides, synthetic glycerides, glycerin esters of fractionated fatty acids, non-surface active esters of fatty acids, fatty acids, cocoa butter, cocoa butter substitutes, hard fat, natural and synthetic waxes, and petrolatum.

12. The formulation according to claim 10 wherein said dispersion vehicle is one or more material(s) selected from the group consisting of hydrogenated vegetable oils, triglycerides, hydrogenated coco-glycerides, mixed glycerides, hydrogenated glycerides, synthetic glycerides, glycerin esters of fractionated fatty acids, propylene glycol diesters of fatty acids, other non-surface active esters of fatty acids, fatty acids, cocoa butter, cocoa butter substitutes, hard fat, natural and synthetic waxes, and petrolatum.

13. The formulation according to claim 12 wherein said dispersion vehicle is one or more material(s) selected from the group consisting of hydrogenated vegetable oils, hard fats, and hydrogenated coco-glycerides.

14. The formulation according to claim 1 wherein said solid or semisolid dispersion vehicle is a mixture of two or more materials.

15. A method of treating a patient comprising administering a therapeutically effective amount of a nanoparticle pharmaceutical formulation according to claim 1 to a patient in need thereof.

16. The method according to claim 15 for the treatment of a neurodegenerative disease or cancer, wherein the poorly soluble drug substance is 7-chloro-N,N,5-trimethyl-4-oxo-3-phenyl-3,5-dihydro-4H-pyridazino[4,5-b]indole-1-acetamide.

17. The method according to claim 15 for the treatment of anxiety, epilepsy, spasticity, or muscle contractures, wherein the poorly soluble drug substance is 6-fluoro-9-methyl-2-phenyl-4-(pyrrolidin-1-ylcarbonyl)-2,9-dihydro-1H-pyrido[3,4-b]indol-1-one.

18. The method according to claim 15 for the treatment or prevention of arrhythmia, wherein the poorly soluble drug substance is isopropyl 2-butyl-3-[4-[3-(dibutylamino)propyl]benzoyl]-1-benzofuran-5-carboxylate fumarate.

19. A process of preparing a nanoparticle formulation according to claim 1 comprising the steps of:
(a) mixing one or more poorly soluble drug substance with a molten dispersion vehicle which is a solid or semi-solid at room temperature, and
(b) media-milling the mixture to form the nanoparticle formulation.

20. The process according to claim 19 comprising the steps of:
(a) heating a solid or semisolid dispersion vehicle to a first temperature range at or above the melting point of said solid or semisolid dispersion vehicle to form a molten dispersion vehicle;
(b) combining one or more poorly soluble drug substance with the molten dispersion vehicle to form a mixture;
(c) media milling the mixture with a plurality of grinding media to form a nanosuspension; and
(d) cooling the nanosuspension to a second temperature range below the melting point of the solid or semi-solid dispersion vehicle to form the nanoparticle formulation.

21. The process according to claim 19 comprising the steps of:
(a) heating a solid or semisolid dispersion vehicle to a first temperature range at or above the melting point of said solid or semisolid dispersion vehicle to form a molten dispersion vehicle;
(b) combining one or more poorly soluble drug substance with the molten dispersion vehicle to form a mixture;
(c) media milling the mixture with a plurality of grinding media to form a nanosuspension;
(d) filling the nanosuspension into capsules; and
(e) cooling the capsules to a second temperature range below the melting point of the solid or semi-solid dispersion vehicle to form the nanoparticle formulation.

22. The process according to claim 19 comprising the steps of:
(a) heating a solid or semisolid dispersion vehicle to a first temperature range at or above the melting point of said solid or semisolid dispersion vehicle to form a molten dispersion vehicle;
(b) combining one or more poorly soluble drug substance with the molten dispersion vehicle to form a mixture;
(c) media milling the mixture with a plurality of grinding media to form a nanosuspension; and
(d) granulating the nanosuspensions onto a non-surface modifying solid excipient to form a solid formulation.
